(19) United States
(12) Patent Application Publication
(10) Pub. No.: US 2004/0024389 A1
(43) Pub. Date: Feb. 5, 2004

(54) Apparatus for determining and ablating the corneal tissue volume necessary for performing a corneal lamellar grafting operation

(76) Inventor: Giuseppe D'Ippolito, Taranto (IT)

Correspondence Address: BLANK ROME LLP, 600 NEW HAMPSHIRE AVENUE, N.W., WASHINGTON, DC, 20037, US

(21) Appl. No.: 10/380,385

(22) PCT Filed: May 18, 2001
(86) PCT No.: PCT/IT01/00248

(30) Foreign Application Priority Data
Sep. 15, 2000 (IT) MI2000A002024

(51) Int. Cl. A61B 18/20

(52) U.S. Cl. 606/5; 606/11

(57) ABSTRACT

An apparatus for determining and ablating the corneal tissue volume necessary for performing a corneal lamellar grafting operation, in a manner optimized for each patient, comprises a central processing unit, to which are operatively connected a corneal pachymeter, of an optical, ultrasonic and the like type, a pupillometer and a photoablative laser, of an excimer, status solid, phantosecond or the like type.

### BACKGROUND OF THE INVENTION

The present invention relates to an apparatus for determining and ablating the corneal tissue volume necessary for performing a corneal lamellar grafting operation, as optimized for each individual patient.

As is known, in a lamellar corneal grafting operation, a portion of corneal tissue having an even thickness and a variable diameter, depending on the amount and location of the different specific pathologies, is conventionally removed.

Usually, the corneal tissue is removed by a surgical instrument called “Krumeich's microkeratome” which, by a planing type of operation removes a corneal disc having a preset diameter and an approximatively even thickness.

### SUMMARY OF THE INVENTION

Thus, the aim of the present invention is to provide such an apparatus which is suitable to mutually coordinate an assembly of apparatus and which is specifically designed for defining, in an unique and optimum manner, the position, area and volume of the corneal tissue to be removed by a laser ablating operation, in order to optimally perform the ablating operation itself.

Within the scope of the above mentioned aim, a main object of the present invention is to provide such an apparatus which is very efficient in operation and which provides accurate values to allow the operator to perform optimally the ablating operation, i.e. in a manner optimized for each individual patient.

Yet another object of the present invention is to provide such an apparatus which, owing to its peculiar constructional features, is very reliable and safe in operation.

Yet another object of the present invention is to provide such an apparatus which can be easily made by using easily available elements and materials.

According to one aspect of the present invention, the above mentioned aim and objects, as well as yet other objects, which will become more apparent hereinafter, are achieved by an apparatus for determining and removing, by an ablating operation, a corneal tissue volume necessary for performing a lamellar corneal grafting operation, as optimized for each individual patient, characterized in that said apparatus comprises a central processing unit, to which are operatively connected a corneal pachymeter, a pupillometer and a photoablative laser.

### BRIEF DESCRIPTION OF THE DRAWINGS

Further characteristics and advantages of the present invention will become more apparent hereinafter from the following disclosure of a preferred, though not exclusive, embodiment of an apparatus for determining and ablating an optimum volume of a corneal tissue as necessary for performing a lamellar corneal grafting operation, which is illustrated, by way of an indicative, but not limitative, example, in the accompanying drawings, where:

FIG. 1 is a flow diagram illustrating the operating flows which can be performed by the apparatus according to the present invention;

FIG. 2 is a cross-sectional view illustrating an optimum patient receiving bed;

FIG. 2a is a further cross sectional view illustrating a typical section of a donor lens;

FIG. 3 illustrates a typical section of the optimum receiving bed;

FIG. 3a represents an optimum typical section of the donor lens;

FIG. 4 illustrates a typical section of the optimum patient ablating contour, according to the present invention;

FIG. 4a represents a typical section of the donor lens ablating contour; and

FIG. 5 represents a block diagram of the ablating apparatus according to the present invention.

### DESCRIPTION OF THE PREFERRED EMBODIMENTS

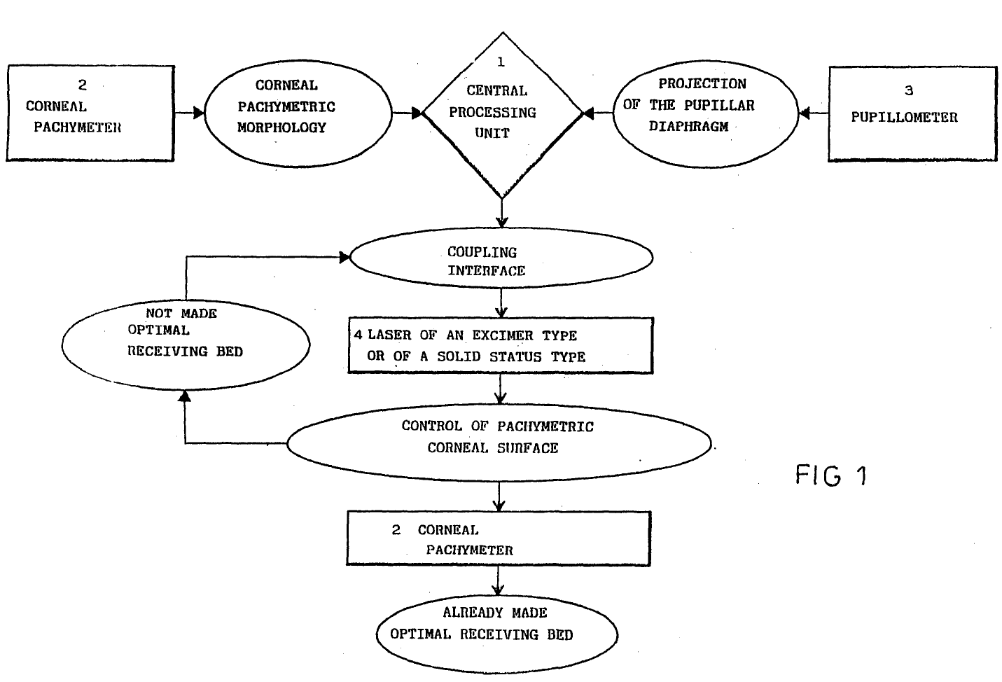

With reference to-the number references of FIG. 1, the apparatus for determining and removing, by an ablating operation, a corneal tissue volume necessary for a lamellar grafting transplantation, as optimized for the individual patient, comprises a central processing unit, generally indicated by the reference number 1, to which are coupled a corneal pachymeter (of an optical, ultrasound or the like type), generally indicated by the reference number 2, provided for morphologically detecting the corneal thickness and for tridimensionally mapping it.

To said central processing unit (1) a pupillometer, indicated by the reference number 3, for determining the projection of the pupillar diaphragm, at the level of the corneal front surface, is connected.

To said central processing unit 1 is moreover connected a photoablative laser (of an excimer, solid status, phantosecond or the like type), of a microspot type, generally indicated by the reference number 4, provided with a coupling interface, for reading the altimetric ablative datum, as expressed by microns on a x, y plane matrix or array.

The apparatus according to the present invention allows to find the ablating volume, which is obtained from a difference of the pachymetric map, as detected for the individual patient by the pachymeter 2, and the optimum pachymetric map of the bed receiving the donor lens.

The operator, in using the apparatus according to the invention, will detect at the start, by using the pupillometer 3, the projection of the pupil on the cornea front surface, the diameter thereof and the position of the related centroid with respect to the corneal limbus, as well as thy diameter of the overall cornea and the position of its centroid, with respect to the pupillar centroid.

Then, the operator will detect the pachymetric data by using a corneal pachymeter (of an optical, ultrasonic or the like type) 2.

As is known, the pachymetric map is the tridimensional contour or profile which is best interpolatedly approximated to the data detected by the corneal pachymeter 2.

The optimum contour or profile of the donor lens receiving bed is defined by the operator by finding or detecting the following parameters:

the center of the receiving bed;

the diameter of the receiving bed;

the minimum thickness at the center of the receiving bed

the maximum thickness at the edge of the receiving bed; and

the thickness variation along the diameter of the receiving bed.

The receiving bed center can be either selected between the corneal centroid, the projection on the cornea of the pupillar centroid, or arbitrarily by the operator.

The diameter of the receiving bed, in turn, is defined by the operator depending on the amount and location of the patient pathology.

The minimum and maximum thicknesses of the receiving bed are defined by the operator depending on the detected pathology analysis and the desired post-surgical refracting geometry.

The variation of the thickness along the receiving bed diameter is, depending on the operator selection, an even variation, a linear or exponential variation, depending on the desired post-surgical refracting geometry.

By using the definition of the above mentioned parameters, the operator will detect, in an unique and optimum manner for the patient, the receiving bed provided for receiving lens of the donor.

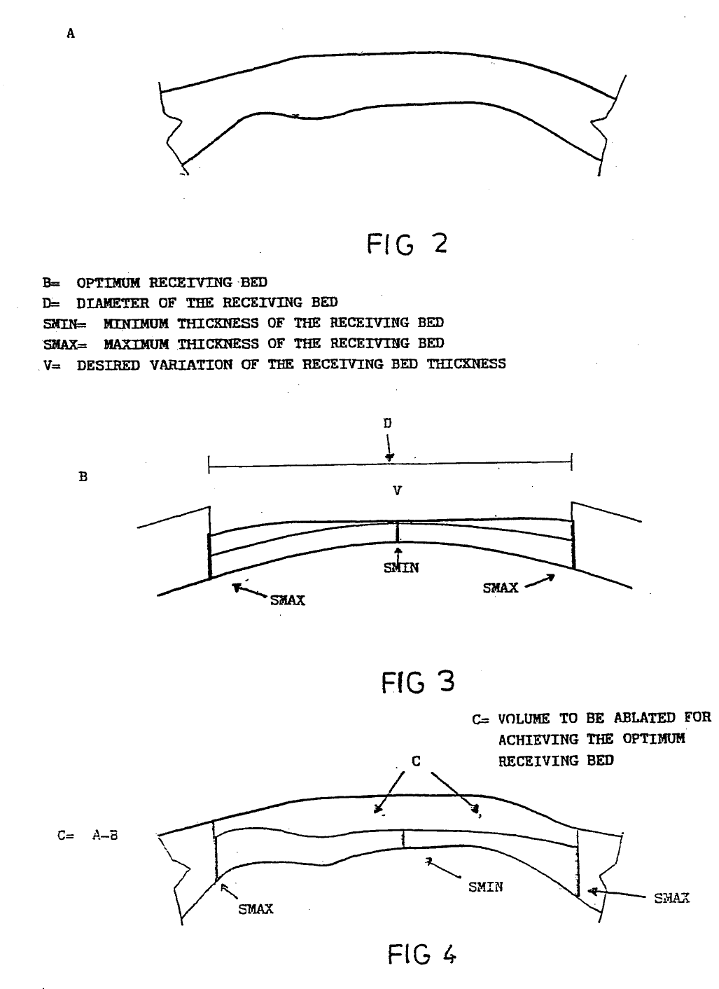

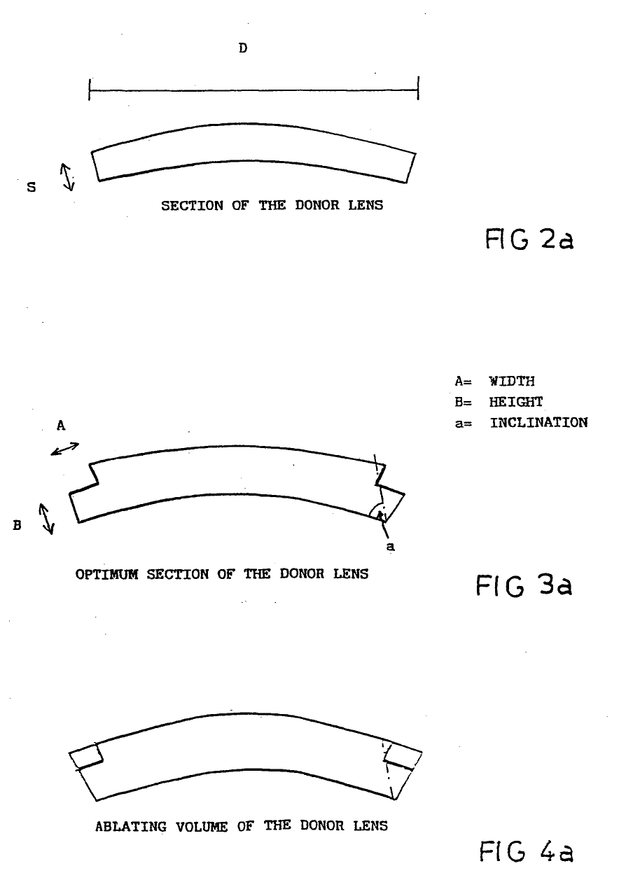

The ablating volume shown in FIG. 2 is then obtained from the difference of the pachymetric map, as determined as above indicated, and the thus defined receiving bed.

The above disclosed method will univocally define the tissue to be ablated volume, in order to set an optimum patient receiving bed.

It is graphically represented by different color areas, clearly showing the receiving bed, its location and the residual cornea area not involved in the operation.

Moreover, digital data will express the overall ablating surface, the overall ablating volume, the maximum and minimum ablating volumes and related planimetric locations as well as the planimetric location of the ablating center with respect to the pupillar centroid.

The ablating volume related to the donor lens is obtained by defining the width A and height B of FIG. 3a to be generated on the lens or, alternately, the desired inclination or slope α.

The altimetric ablative datum is represented on a x, y-plane square matrix, to allow the photoablative laser 4 to properly perform its detection operation, through a suitable interface, for detecting the ablating contour or profile which, upon detection, is practically followed.

The system, moreover, optionally through an intra-operating detection of the pachymetric datum, will verify that the treatment has been carried out according to the programmed ablating strategy, while modifying the number of surface-unit localized pulses.

In this case, the operation will end upon achieving a congruency of the detected and desired data.

It should be pointed out that FIG. 3 clearly shows the optimum receiving bed B, the letter D showing the diameter of said receiving bed.

In this figure, Smin shows the minimum thickness of the receiving bed, whereas Smax shows the maximum thickness of said receiving bed.

The letter V shows, in turn, the desired variation of the receiving bed thickness.

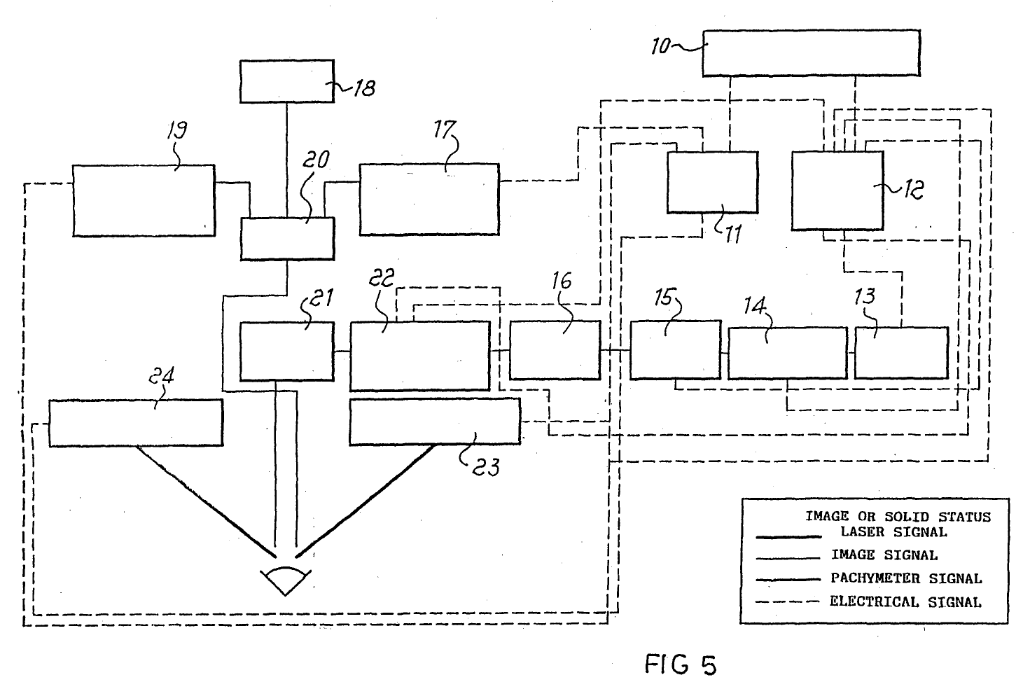

In FIG. 4, the letter C shows the volume to be ablated for achieving the optimum receiving bed.

In this figure, moreover, Smax and Smin show respectively the maximum and minimum thickness of the mentioned receiving bed.

With reference to FIG. 5, which is a block diagram of the apparatus according to the invention, said apparatus comprises a central processing unit 10, which is coupled to a pachymetric control unit 11 and a photoablative laser control unit 12.

The latter is coupled to a laser cavity 13, fitted, in turn, to a control device for controlling the power of the laser beam 14 and to a device 15 for measuring the laser beam power.

This apparatus is moreover characterized in that it further comprises a focalizing system 16 for focalizing the laser beam, coupled to a galvanometric system 22 and to an orienting system 21 for orienting the laser beam.

Moreover, a system 23 for detecting the corneal pachymetry and a source 24 for detecting said corneal pachymetry are moreover provided.

The apparatus comprises furthermore an optic divider 20, to divide or split the video signal between an operating microscope 18, a video camera 19 for detecting the ocular motility, coupled to the photoablative laser control unit 12 and a further video camera 17 for detecting the ocular motility, coupled to the pachymeter control unit 11.

From the above disclosure it should be apparent that the invention fully achieves the intended aim and objects.

In particular, an apparatus according to the block diagram shown in FIG. 5 has been provided, which is adapted to allow to detect in a very accurate manner the data and/or parameters necessary for addressing and properly performing the surgical operation.

The invention, as disclosed, is susceptible to several modifications and variations, all of which will come within the scope of the invention.

Moreover, the used materials, as well as the contingent size and shapes, can be any, depending on requirements.

1. An apparatus for determining and removing, by an ablating operation, a corneal tissue volume necessary for performing a lamellar corneal grafting operation, as optimized for each individual patient, comprising a central processing unit, to which are operatively connected a corneal pachymeter, a pupillometer and a microspot photoablative laser controlled by a microspot photoablative laser control unit, characterized in that said corneal pachymeter is designed for performing a morphologic detection and a tridimensional mapping of the cornea thickness, said pupillometer is designed for determining the projection of the pupillar diaphragm at the level of the cornea front surface, the diameter thereof and the location of the centroid thereof, with respect to a corneal limbus, and the diameter of the overall cornea and the location of the centroid thereof with respect to the pupillar centroid, and that said microspot ablative laser comprises a laser cavity to which said microspot ablative laser control unit is coupled, said cavity being in turn coupled to a control device for controlling the power of the microspot laser beam and to a device for measuring the power of the microspot laser beam.

2. An apparatus according to claim 1, characterized in that said microspot photoablative laser is coupled to said central processing unit through an interface and is controlled by said central processing unit so as to read an altimetric ablating datum expressed in microns on a x, y-plan cartesian matrix.

3. An apparatus according to claims 1 and 2, characterized in that said ablation volume is obtained from a difference of a pachymetric map detected for an individual patient by said pachymeter and an optimum pachymetric map of the eye lens receiving bed.

4. An apparatus according to claims 1 to 3, characterized in that said central processing unit determines an optimum contour of the donor eye lens receiving bed by processing a series of parameters including the center of said receiving bed, the diameter of said receiving bed, the minimum thickness at the center of said receiving bed, the maximum thickness at the edge of said receiving bed and a thickness variation along the diameter of said receiving bed.

5. An apparatus according to claim 4, characterized in that said center of said receiving bed is either selected between the corneal centroid, the projection of the pupillar centroid on the cornea or arbitrarily by the operator.

6. An apparatus according to claim 4, characterized in that said diameter of said receiving bed is determined depending on the amount and location of the patient detected pathology.

7. An apparatus according to claim 1, characterized in that said central processing unit provides digital data indicating the overall ablating surface, the overall ablating volume, the ablating maximum and minimum thicknesses and related planimetric offset or dislocation, and the planimetric offset r dislocati n of the ablating cent r from the pupillar centroid.

8. An apparatus according to claim 1, characterized in that said apparatus further comprises a focalizing system for focalizing said laser beam, said focalizing system being coupled to an orienting galvanometric system for orienting the laser beam and to a laser beam orienting mirror system.

9. An apparatus according to claim 1, characterized in that said apparatus further comprises an operating microscope coupled to an optic divider, in turn connected to a video camera for detecting the ocular motility.

10. An apparatus according to claims 1 and 9, characterized in that said apparatus comprises a pachymetric control unit coupled to said video camera for detecting said ocular motility.

11. An apparatus according to claim 1, characterized in that said apparatus further comprises an orienting mirror system, a corneal pachymetry detecting system and a corneal pachymetry detecting source.
